(19) United States
(12) Patent Application Publication
(10) Pub. No.: US 2009/0186859 A1
(43) Pub. Date: Jul. 23, 2009

(54) DIAZENIUMDIOLATED NON-STEROIDAL ANTI-INFLAMMATORY DRUGS, COMPOSITIONS THEREOF, AND RELATED METHODS

(75) Inventors: Carlos A. Velázquez, Frederick, MD (US); Joseph E. Saavedra, Thurmont, MD (US); Larry Keefer, Bethesda, MD (US); Brett Showalter, New Market, MD (US); Edward E. Knaus, Edmonton (CA)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignees: THE UNITED STATES OF AMERICA, AS REPRESENTED BY THE SEC., DEPT. OF HEALTH AND HUMAN SERVICES, Bethesda (MD); The Governors of the University of Alberta, Edmonton, Alberta (CA)

(21) Appl. No.: 12/298,322

(22) PCT Filed: Apr. 24, 2007
(86) PCT No.: PCT/US07/67295

§ 371 Date: Jan. 15, 2009

Related U.S. Application Data
(60) Provisional application No. 60/794,421, filed on Apr. 24, 2006.

(51) Int. Cl. A61K 31/655; C07C 245/24; A61P 19/02

(52) U.S. Cl. 514/149; 534/551

(57) ABSTRACT

Disclosed are compounds that release nitric oxide, e.g., a compound of Formula (I) wherein R¹⁻¹⁰, X, and n are as described herein, which are NSAID derivatives comprising a diazeniumdiolate moiety N₂O₂⁻. The compounds are chemopreventive agents with gastric-sparing, analgesic, cardioprotective, and/or anti-inflammatory properties. Also disclosed is a pharmaceutical composition comprising a compound of the invention and a pharmaceutically acceptable carrier. Also disclosed is a method of preventing or treating cancer or treating inflammation or an inflammation-related condition in a mammal comprising administering an effective amount of a compound of the invention to the mammal.

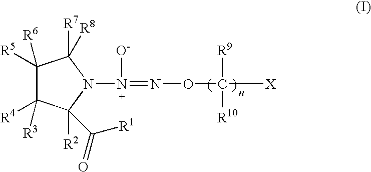

### CROSS-REFERENCE TO A RELATED APPLICATION

This patent application claims the benefit of U.S. Provisional Patent Application No. 60/794,421, filed Apr. 24, 2006, the disclosure of which is incorporated by reference.

### BACKGROUND OF THE INVENTION

Non-steroidal anti-inflammatory drugs (NSAIDs) constitute the main class of drugs used clinically to treat pain and inflammation. Classical NSAIDs have been prescribed worldwide for more than five decades to treat the untoward consequences of acute and chronic inflammatory conditions, such as arthritis and osteoarthritis. Furthermore, recent epidemiological studies have shown that NSAIDs reduce the risk of, and mortality from, colorectal cancer (CRC), by about half and constitute the prototypical colon cancer chemopreventive agents (Shiff et al., Gastroenterology 1997, 113, 1992-1998).

However, the prolonged use of NSAIDs is limited by their significant toxicity, which includes gastrointestinal (dyspepsia, bleeding, obstruction and perforation) and renal side-effects, hypersensitivity reactions and salicylate intoxication (Shiff et al., Gastroenterology 1997, 113, 1992-1998 and Kashfi et al., Biochem. Soc. Trans. 2005, 33, 701-704). Indeed, in 1998 as many people died in the US from NSAID-induced complications as from AIDS (Singh et al., J. Rheumatol. Suppl 1999, 56, 18-24).

The practice of cancer chemoprevention involves the administration of a chemical or a naturally occurring agent to individuals at high risk, to prevent the development or recurrence of cancer. Therefore, a successful chemopreventive agent should meet one or more criteria: it must be effective, devoid of significant side-effects, and convenient to administer. Since a chemopreventive agent will be administered to individuals at risk of developing cancer for many years, the criteria of high efficiency and safety are of particular importance (Kashfi et al., Biochem. Soc. Trans. 2005, 33, 701-704). These considerations have prompted intensive efforts to identify alternatives.

### BRIEF SUMMARY OF THE INVENTION

The invention provides novel compounds which are NSAID derivatives comprising a functional portion of NSAID, a diazeniumdiolate moiety N₂O₂⁻, and a 2-substituted pyrrolidin-1-yl moiety, for example compounds of Formulas I and II described below. The compounds of the invention release nitric oxide under physiological conditions and have one or more of the following advantages. They do not require a metabolically demanding redox reaction to release NO, they contain twice as much available NO per NO-releasing functional group as organic nitrate-based NO-NSAIDs, they are hybrids of naturally occurring amino acid L-proline, whose N-nitroso derivative is not toxic or carcinogenic, they can generate free NSAID and cytoprotective NO simultaneously in one activation step rather than two, and they are not expected to induce nitrate tolerance. The compounds of the invention are non-toxic, tolerance free chemopreventive agents with gastric-sparing, analgesic, cardioprotective, and/or anti-inflammatory properties.

The invention further provides pharmaceutical compositions comprising at least one compound of the invention and a pharmaceutically acceptable carrier.

The invention also provides a method of preventing or treating cancer in a mammal comprising administering an effective amount of at least one compound of the invention to the mammal.

### BRIEF DESCRIPTION OF THE SEVERAL VIEWS OF THE DRAWING(S)

FIG. 1 outlines a method for preparing intermediates (13-15) in the synthesis of a compound in accordance with an embodiment of the invention. Step a: NO, ether, CH₃ONa. Step b: R—X, DMF or DMSO. Step c: NaIO₄, RuCl₃, H₂O/CH₃CN/EtOAc.

FIG. 2 outlines a method for synthesis of O²-(acetoxymethyl) 1-[2-(carboxylato)pyrrolidin-1-yl)diazen-1-ium-1,2-diolate (18), an intermediate in the synthesis of compound 20a, in which the NSAID is aspirin, in accordance with an embodiment of the present invention.

FIG. 3 outlines a method for synthesis of compound 20a, wherein the NSAID is represented by acetyl salicylic acid (21). Step a: NO (40 psi), CH₃ONa/CH₃OH, ether, r.t., 48 h. Step b: BrCH₂OCOCH₃, Na₂CO₃, DMSO, r.t., 15 h. Step c: RuCl₃, NaIO₄, H₂O, CH₃CN, EtOAc, r.t., 2 h. Step d: TEA, DMSO, r.t., 24 h. Step e: ClSO₃CH₂C₁, H₂O, DCM, NaHCO₃, Bu₄NHSO₄, r.t., 30 min.

FIG. 4 outlines a method for synthesis of compound 28 in accordance with an embodiment of the invention, wherein the NSAID is acetyl salicylic acid (21). Step a: NO, CH₃ONa. Step b: ClCH₂SCH₃, DMF. Step c: THF, TEA. Step d: SO₂Cl₂, DCM. Step e: DMSO, TEA. Step f: CH₃CN/H₂O, K₂CO₃ (cat.). Step g: NaIO₄, RuCl₃, DCM/H₂O, EA.

FIG. 5 outlines a method for synthesis of compound 31 in accordance with an embodiment of the invention, wherein the NSAID is acetyl salicylic acid (21). Step a: NO, CH₃ONa. Step b: DNCB, DMF. Step c: NaIO₄, RuCl₃, DCM/H₂O, EA. Step d: DMSO, TEA.

### DETAILED DESCRIPTION OF THE INVENTION

The present invention tales advantage of the fact that, unlike organic nitrates, O₂-unsubstitued N-diazen-1-ium-1,2-diolates (NONOates, 1, see Equation 1 below) dissociate spontaneously in phosphate buffer solution (PBS) at 37° C. to regenerate up to 2 equivalents of .NO and the corresponding secondary amine (2), with a wide variety of half-lives (from 2 seconds to several days). NONOates are minimally affected by metabolism, and are essentially different from currently available clinical vasodilators that require redox activation before .NO is released (Keefer et al., Annu. Rev. Pharmacol. Toxicol. 2003, 43, 585-607). N-diazeniumdiolates possess three attributes that make them especially attractive for designing drugs to treat a variety of disease states, namely structural diversity, dependable rates of .NO release, and rich O²-derivatization chemistry that facilitates targeting of .NO to specific target organ and/or tissue sites (Keefer, supra).

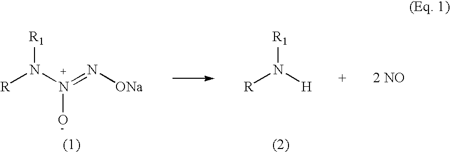

The present invention provides, in an embodiment, a compound of Formula I

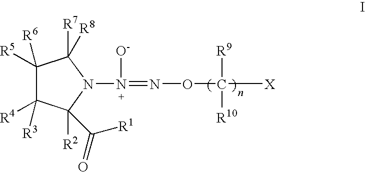

wherein

R¹ is hydrogen, OH, halo, NR¹¹R¹², OR¹¹, SR¹¹, or OM_{1/m}, wherein M is a cation and

m is the valency of M (e.g., m is 1-6),

R², R⁷, and R⁸ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, carboxy, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, or nitro;

R³⁻⁶ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, hydroxy, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, mercapto, an unsubstituted or substituted C₁₋₁₂ alkylthio, amino, an unsubstituted or substituted C₁₋₁₂ alkylamino, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, halo, or nitro;

R⁹ and R¹⁰ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, an unsubstituted or substituted heteroarylamino, an unsubstituted or substituted heteroaryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, or benzylcarbonyl;

X is a functional portion of an NSAID;

R¹¹ and R¹² are independently hydrogen, an unsubstituted or substituted C₁₋₁₂ alkyl, an unsubstituted or substituted C₂₋₁₂ alkenyl, an unsubstituted or substituted C₃₋₃₀ cycloalkyl, an unsubstituted or substituted C₆₋₃₀ aryl, an unsubstituted or substituted heteroaryl, or an unsubstituted or substituted heterocyclyl; and

n is 0-3.

The counterion, M, is any pharmaceutically acceptable counterion, which could be a metal or non-metal counterion, e.g., alkali metal counterions such as sodium ion, potassium ion, lithium ion, and the like; alkaline earth metal counterions such as magnesium ion, calcium ion, and the like; Group III metal counterions such as aluminum ion; Group IV metal counterions such as tin ion; and transition metals, including iron ion, copper ion, manganese ion, zinc ion, cobalt ion, vanadium ion, molybdenum ion, platinum ion, and the like. Non-metal counterions include quaternary ammonium ions.

Preferably R¹ is hydrogen, OH, OR¹¹, or OM₁₋ₘ. Preferably, R², R⁷, and R⁸ are individually selected from hydrogen, an unsubstituted or substituted C₁₋₁₂ alkyl, an unsubstituted or substituted C₆₋₃₀ aryl, and an unsubstituted or substituted C₁₋₁₂ alkoxy. Preferably, R³⁻⁶ are individually selected from hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo. Preferably, R⁹ and R¹⁰ are individually selected from hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl. In preferred compounds of Formula I, n is 1. In one embodiment, a compound of Formula I includes where the NSAID is aspirin, ibuprofen, sulindac, indomethacin, or celecoxib.

In a further embodiment, a compound of Formula I includes where R¹ is hydrogen, OH, OR¹¹, or OM_{1/m}; R², R⁷, and R⁸ are individually selected from hydrogen, an unsubstituted or substituted C₁₋₁₂ alkyl, an unsubstituted or substituted C₆₋₃₀ aryl, and an unsubstituted or substituted C₁₋₁₂ alkoxy; R³⁻⁶ are individually selected from hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo; R⁹ and R¹⁰ are individually selected from hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl; and the NSAID is aspirin, ibuprofen, sulindac, indomethacin, or celecoxib. In another embodiment, a compound of Formula I includes R¹ is OH, R²⁻⁸ are hydrogen, R⁹ and R¹⁰ are hydrogen and phenyl, respectively, and n is 1. Another preferred compound includes where one of R⁵ or R⁶ is OH, and the other is hydrogen, R²⁻⁴ and R⁷⁻¹⁰ are hydrogen, and the NSAID is aspirin, ibuprofen, sulindac, indomethacin, or celecoxib. Another preferred compound is where R¹ is OH, R²⁻¹⁰ are hydrogen, n is 1, and the NSAID is aspirin (28):

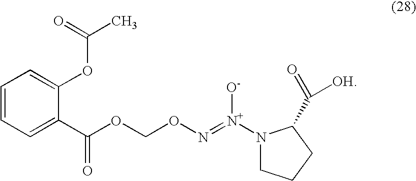

Additional examples of compounds of Formula I include (where the NSAID is celecoxib)

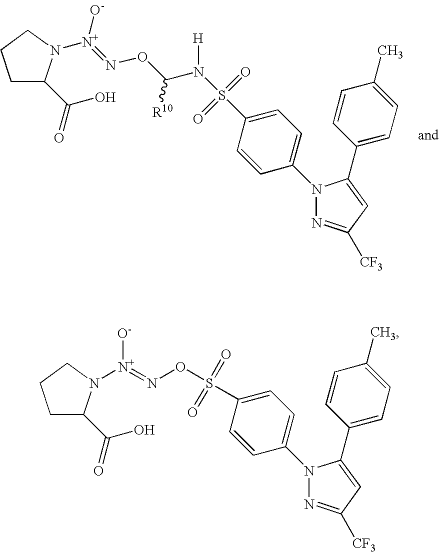

wherein R¹⁰ is hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, or an unsubstituted or substituted heterocyclyl.

The present invention provides, in another embodiment, a compound of Formula II

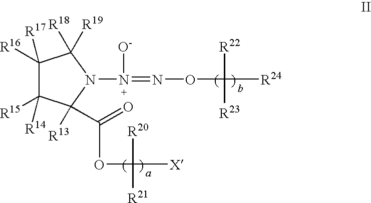

wherein

X′ is a functional portion of an NSAID;

R¹³, R¹⁸, and R¹⁹ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, carboxy, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, or nitro;

R¹⁴⁻¹⁷ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, hydroxy, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, mercapto, an unsubstituted or substituted C₁₋₁₂ alkylthio, amino, an unsubstituted or substituted C₁₋₁₂ alkylamino, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, halo, or nitro;

R²⁰⁻²³ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, an unsubstituted or substituted heteroarylamino, an unsubstituted or substituted heteroaryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, or benzylcarbonyl;

R²⁴ is an unsubstituted or substituted C₁₋₁₂ acyloxy, an unsubstituted or substituted carboxamido, an unsubstituted or substituted C₁₋₁₂ alkyl, an unsubstituted or substituted C₂₋₁₂ alkenyl, an unsubstituted or substituted C₃₋₃₀ cycloalkyl, an unsubstituted or substituted C₆₋₃₀ aryl, or an unsubstituted or substituted C₁₋₁₂ alkoxy-C₁₋₁₂ alkyl; and

a and b are independently 0-3.

Preferably, R¹³, R¹⁸, and R¹⁹ are individually selected from hydrogen, an unsubstituted or substituted C₁₋₁₂ alkyl, an unsubstituted or substituted C₆₋₃₀ aryl, and an unsubstituted or substituted C₁₋₁₂ alkoxy. Preferably, R¹⁴⁻¹⁷ are individually selected from hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo. Preferably, R²⁰⁻²³ are individually selected from hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl. Preferably, R²⁴ is an unsubstituted or substituted C₁₋₁₂ acyloxy, an unsubstituted or substituted C₁₋₁₂ alkyl, an unsubstituted or substituted C₆₋₃₀ aryl, or an unsubstituted or substituted C₁₋₁₂ alkoxy-C₁₋₁₂ alkyl. In preferred compounds of Formula II, a and b are each 1. In one embodiment, a compound of Formula II includes where the NSAID is aspirin, ibuprofen, sulindac, indomethacin, or celecoxib.

In a further embodiment, a compound of Formula II includes where R¹³, R¹⁸, and R¹⁹ are individually selected from the group consisting of hydrogen, an unsubstituted or substituted C₁₋₁₂ alkyl, an unsubstituted or substituted C₆₋₃₀ aryl, and an unsubstituted or substituted C₁₋₁₂ alkoxy; R¹⁴⁻¹⁷ are individually selected from the group consisting of hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo; R²⁰⁻²³ are individually selected from the group consisting of hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl; R²⁴ is an unsubstituted or substituted C₁₋₁₂ acyloxy, an unsubstituted or substituted C₁₋₁₂ alkyl, an unsubstituted or substituted C₆₋₃₀ aryl, or an unsubstituted or substituted C₁₋₁₂ alkoxy-C₁₋₁₂ alkyl; and the NSAID is aspirin, ibuprofen, sulindac, indomethacin, or celecoxib. In another embodiment, a compound of Formula II includes R¹³⁻¹⁹ are hydrogen, R²⁰⁻²³ are individually hydrogen, methyl, ethyl, i-propyl, t-butyl, or phenyl, R²⁴ is an unsubstituted or substituted C₁₋₁₂ acyloxy, a and b are each 1, and the NSAID is aspirin, ibuprofen, sulindac, indomethacin, or celecoxib. A preferred compound includes where R¹³⁻²³ are hydrogen, R²⁴ is acetoxy or benzoyloxy, and the NSAID is aspirin. Another preferred compound includes where R¹³⁻²³ are hydrogen, R²⁴ is acetoxy or benzoyloxy, and the NSAID is indomethacin. Another preferred compound includes where one of R¹⁶ or R¹⁷ is OH, and the other is hydrogen, R¹³⁻¹⁵ and R¹⁸⁻²³ are hydrogen, R²⁴ is acyloxy or arylcarboxy, and the NSAID is aspirin, ibuprofen, sulindac, indomethacin, or celecoxib. Other preferred compounds include where R¹³⁻²¹ are hydrogen, R²⁴ is an unsubstituted or substituted C₆₋₃₀ aryl (e.g., 2,4-dinitrophenyl), a is 1, b is 0, and the NSAID is aspirin or indomethacin, as in structures 31 and 32, respectively.

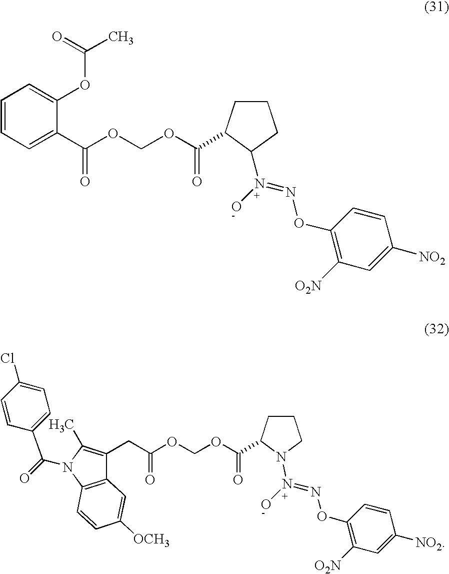

The compound of Formula I or II can be chiral or achiral. If the compound is chiral, it can be the R enantiomer, the S enantiomer, or a mixture of both (including a racemic mixture). If more than one chiral center is present, the stereoisomers of the compound of Formula I or II can be diastereomers of one another and can include a meso compound.

As regards the term “substituted” referring to the various groups in Formula I and II, any one of the groups that can be substituted, wherever possible, generally can have 1 to 10 substituents (e.g., 1 to 8, 1 to 6, 1 to 4, 1 to 3 substituents) that are independently selected from the group consisting of C₁₋₁₂ alkyl, C₃₋₃₀ cycloalkyl, C₆₋₃₀ aryl, heteroaryl, C₁₋₁₂ alkoxy, C₁₋₁₂ aryloxy, acyloxy, formyl, acetyl, carboxyl, carboxy-C₁₋₁₂ alkyl, carboxy-C₁₋₁₂ alkylamido, carboxy-C₁₋₁₂ dialkylamido, carboxamido, C₁₋₁₂ alkylcarbonyl, C₆₋₃₀ arylamino, C₆₋₃₀ diarylamino, nitrile, phenylcarbonyl, benzylcarbonyl, halo, cyano, hydroxy, mercapto, nitro, amino, C₁₋₁₂ alkylamino, and C₁₋₁₂ dialkylamino.

Referring now to terminology used generically herein, the term “alkyl” implies a straight-chain or branched alkyl substituent containing from, for example, about 1 to about 12 carbon atoms, preferably from about 1 to about 8 carbon atoms, more preferably from about 1 to about 6 carbon atoms. Examples of such substituents include methyl, ethyl, propyl, isopropyl, n-butyl, sec-butyl, isobutyl, tert-butyl, pentyl, isoamyl, hexyl, octyl, dodecanyl, and the like.

The term “alkenyl,” as used herein, means a linear alkenyl substituent containing from, for example, about 2 to about 12 carbon atoms (branched alkenyls are about 3 to about 12 carbons atoms), preferably from about 2 to about 8 carbon atoms (branched alkenyls are preferably from about 3 to about 8 carbon atoms), more preferably from about 3 to about 6 carbon atoms. Examples of such substituents include propenyl, isopropenyl, n-butenyl, sec-butenyl, isobutenyl, pentenyl, isopentenyl, hexenyl, octenyl, dodecenyl, and the like.

The term “alkynyl,” as used herein, means a linear alkynyl substituent containing at least one carbon-carbon triple bond and from, for example, about 2 to about 12 carbon atoms (branched alkynyls are about 4 to about 12 carbons atoms), preferably from about 2 to about 8 carbon atoms (branched alkynyls are preferably from about 4 to about 8 carbon atoms), more preferably from about 3 to about 6 carbon atoms. Examples of such substituents include propynyl, propargyl, n-butynyl, pentynyl, isopentynyl, hexynyl, octynyl, dodecynyl, and the like.

The term “cycloalkyl,” as used herein, means a cyclic alkyl substituent containing from, for example, about 3 to about 30 carbon atoms, preferably about 3 to about 8 carbon atoms, preferably from about 5 to about 8 carbon atoms, more preferably from about 5 to about 6 carbon atoms. Examples of such substituents include cyclopropyl, cyclobutyl, cyclopentyl, cyclohexyl, and the like.

The term “aryl” refers to an unsubstituted or substituted aromatic carbocyclic substituent, as commonly understood in the art, and includes monocyclic and polycyclic aromatics such as, for example, phenyl, biphenyl, toluenyl, anisolyl, naphthyl, anthracenyl and the like. An aryl substituent generally contains from, for example, about 6 to about 30 carbon atoms, preferably from about 6 to about 18 carbon atoms, more preferably from about 6 to about 14 carbon atoms and most preferably from about 6 to about 10 carbon atoms. It is understood that the term aryl applies to cyclic substituents that are planar and comprise 4n+2 π electrons, according to Hückel's Rule.

The term “heterocyclyl” means a stable, saturated, or partially unsaturated monocyclic, bicyclic, and spiro ring system containing 3 to 7 ring members of carbon atoms and other atoms selected from nitrogen, sulfur, and/or oxygen. Preferably, a heterocyclyl is a 5 or 6-membered monocyclic ring and contains one, two, or three heteroatoms selected from nitrogen, oxygen, and/or sulfur. The heterocyclyl may be attached to the parent structure through a carbon atom or through any heteroatom of the heterocyclyl that results in a stable structure. Examples of such heterocyclic rings are isoxazolyl, thiazolinyl, imidazolidinyl, pyrrolyl, pyrrolinyl, pyrazolyl, pyranyl, piperidyl, oxazolyl, and morpholinyl.

The term “heteroaryl” refers to aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furyl, thienyl, isothiazolyl, thiazolyl, isoxazolyl, and oxadiazolyl.

The term “aralkyl” as utilized herein means alkyl as defined herein, wherein at least one hydrogen atom is replaced with an aryl substituent as defined herein. Aralkyls include, for example, benzyl, phenethyl, and substituents of the formula:

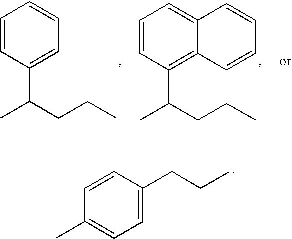

The term “alkoxy” embraces linear or branched alkyl groups that are attached to divalent oxygen. The alkyl group is the same as described herein. Examples of such substituents include methoxy, ethoxy, t-butoxy, and the like. The term “aryloxy” refers to substituents that have an aryl group attached to divalent oxygen. The aryl group is the same as described herein. Examples of such substituents include phenoxy. The term “heteroaryloxy” refers to substituents that have a heteroaryl group attached to divalent oxygen. The heteroaryl group is the same as described herein.

The term “alkylthio” as used herein, denotes a substituent with an alkyl group directly attached to a divalent sulfur atom. The alkyl group is the same as described herein. Examples of such substituents include methylthio, ethylthio, and the like.

The terms “alkylamino” and “arylamino” refer to a secondary amine substituent with one hydrogen and one alkyl or aryl group, respectively, directly attached to a trivalent nitrogen atom. The terms “dialkylamino” and “diarylamino” refer to a tertiary amine substituent with two of the same or different alkyl or aryl groups, respectively, directly attached to a trivalent nitrogen atom. The term “arylalkylamino” refers to a tertiary amine substituent with one aryl substituent and one alkyl substituent. The alkyl and aryl groups are the same as described herein.

The term “halo” or “halogen,” as used herein, means a substituent selected from Group VIIA, such as, for example, fluorine, bromine, chlorine, and iodine. Preferably, the halo is bromine or chlorine.

The term “carboxy” refers to the group —C(O)OH. The term “carboxyalkyl” refers to the group —RC(O)OH that is connected to the compound through the alkyl R group. The term “acyloxy” refers to the group —OC(O)R, in which R is an alkyl group as described herein.

The term “carboxyalkylamino” refers to the group —NHRC(O)OH, in which R is an alkyl (e.g., (CH₂)ₙ alkylene group, n is 1 to 12) group. The term “carboxydialkylamino” refers to the group —NR′RC(O)OH, in which R is a (CH₂), alkylene group (n is 1 to 12) and R′ is an alkyl group as described herein.

The term “carboxamido” refers to the group —C(O)NH₂. The term “alkylcarboxamido” refers to the group —C(O)NHR, which R is an alkyl group as described herein. The term “dialkylcarboxamido” refers to the group —C(O)NRR′, which R and R′ are the same or different and are alkyl groups as described herein.

The term “alkylcarbonyl” refers to the group —C(O)R, in which R is an alkyl group as described herein. The term “aroyl” refers to the group —C(O)Ar, in which Ar is an aryl group as described herein.

The term “cyclooxygenase (COX) inhibitor” refers to a compound that inhibits any cyclooxygenase enzyme, including, but not limited to cyclooxygenase-1 enzyme, cyclooxygenase-2 enzyme and/or cyclooxygenase-3 enzyme and mixtures of two or more thereof. “COX inhibitors” include, for example, NSAIDs, cyclooxygenase-1 (COX-1) selective inhibitors, cyclooxygenase-2 (COX-2) selective inhibitors, and cyclooxygenase-3 (COX-3) selective inhibitors.

The term “cyclooxygenase-2 (COX-2) selective inhibitor” refers to a compound that selectively inhibits the cyclooxygenase-2 enzyme over the cyclooxygenase-1 enzyme.

The term “NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

The chemical structures of NSAIDs vary. Some NSAIDs are based on salicylic acid and include, for example, aspirin (e.g., acetylsalicylic acid), salicylate esters and salts, acetate esters of salicylic acid, difluorophenyl derivatives (e.g., diflunisal), salicylsalicylic acids (e.g., salsalate), salts of salicylic acids (e.g., sodium salicylate), salicylamide, sodium thiosalicylate, choline salicylate, magnesium salicylate, 5-aminosalicylic acid (e.g., mesalamine), salicylazosulfapyridine (e.g., sulfasalazine), and methylsalicylate.

Another group of NSAIDs are the pyrazolon derivatives, which include, for example, phenylbutazone, oxyphenbutazone, antipyrine, aminopyrine, dipyrone and apazone (azapropazone). Another group of NSAIDs are the para-aminophenol derivatives, which are the so-called “coal tar” analgesics, including, for example, phenacetin and its active metabolite acetaminophen. Yet another group of compounds is the fenamates which are derivatives of N-phenylanthranilic acid (e.g., mefenamic, meclofenamic, flufenamic, tolfenamic and etofenamic acids). Another group of NSAIDs is the propionic acid derivatives, which includes, for example, ibuprofen, naproxen, flurbiprofen, fenoprofen, ketoprofen, fenbufen, pirprofen, oxaprozin, indoprofen, and tiaprofenic acid.

Still other NSAIDs are a class of antiinflammatory enolic acids (e.g., piroxicam, ampiroxicam, meloxicam, tenoxicam, tenidap and oxicam derivatives), phenylacetic acid derivatives (e.g., diclofenac), and cyclized derivatives of arylpropionic acids, arylacetic acids, and thiazinecarboxamides.

Suitable NSAIDs include, but are not limited to, acetaminophen, acemetacin, aceclofenac, alminoprofen, amfenac, aminopyrine, ampiroxicam, antipyrine, apazone, aspirin, bendazac, benoxaprofen, bromfenac, bucloxic acid, bumadizon, butibufen, carprofen, celecoxib, choline salicylate, cinmetacin, clidanac, clopirac, diclofenac, diflunisal, dipyrone, enfenamic acid, etodolac, etofenamic acid, felbinac, fenbufen, fenclozic acid, fendosal, fenoprofen, fentiazac, flufenamic acid, flunixin, flunoxaprofen, flurbiprofen, gentisic acid, ketorolac, ibufenac, ibuprofen, indomethacin, indoprofen, isofezolac, isoxepac, indoprofen, ketoprofen, lonazolac, loxoprofen, magnesium salicylate, meclofenamic acid, methylsalicylate, mefenamic acid, mesalamine, metiazinic acid, mobicox, mofezolac, miroprofen, nambumetone, naproxen, nemisulide, oxaprozin, oxyphenbutazone, phenylbutazone, piroxicam, pirozolac, pirprofen, pranoprofen, protizinic acid, rofecoxib, salsalate, sodium salicylate, sodium thiosalicylate, salicylamide, sulfasalazine, sulindac, suprofen, suxibuzone, tenoxicam, tenidap, tiaprofenic acid, tolfenamic acid, tolmetin, xenbucin, ximoprofen, valdecoxib, zaltoprofen, zomepirac, and the like. Suitable NSAIDs are described more fully in the literature, such as in Goodman and Gilman, The Pharmacological Basis of Therapeutics (9th Edition), 15 McGraw-Hill, 1995; the Merck Index on CD-ROM, 13th Edition; and in U.S. Pat. Nos. 6,057,347 and 6,297,260.

Preferred NSAIDs include, but are not limited to, aspirin, ibuprofen, indomethacin, sulindac, ketoprofen, fenoprofen, flurbiprofen, naproxen, nambumetone, nemisulide, piroxicam, tiaprofenic acid, suprofen, etodolac, carprofen, ketorolac, pirprofen, indoprofen, celecoxib, rofecoxib, valdecoxib, mobicox, and benoxaprofen.

The compounds of Formula I and II can be prepared by any suitable method. For example, the substituted pyrrolidinyl group can be diazeniumdiolated first, and the functional portion of NSAID can be attached through a linker on the O₂-oxygen on the N₂O₂⁻ moiety. Alternatively, the functional portion of NSAID can be bonded to the pyrrolidinyl group first and then exposed to nitric oxide to form the diazeniumdiolate group. For example, a substituted pyrrolidinyl group, such as prolinol (12) is reacted with .NO in the presence of a base to yield the corresponding diazeniumdiolate. The diazeniumdiolate is reacted with an electrophile (R-X) to provide an O²-protected diazeniumdiolate. If necessary, the substituent(s) on the pyrrolidinyl group can be further modified (e.g., oxidized). The O²-protected diazeniumdiolate can then be reacted under suitable conditions (e.g., basic) for attaching the desired NSAID moiety to provide a compound of either Formula I or II.

The functional portion of the NSAID (or X or X′) can be attached to the rest of the compound of Formula I or II by any suitable linkage. The functional portion (X or X′) is the portion of the NSAID molecule that retains the pharmacological activity. The NSAID molecule can be bonded to the diazeniumdiolate moiety without a chemical modification to the NSAID or with a suitable modification. For example, if an NSAID is bonded tluough a carboxyl (—COOH) moiety present on the NSAID, then X or X′ would be the NSAID molecule minus OH (or minus H if O is retained) if the OH group of the carboxyl was lost during the bonding reaction. The NSAID also can be bonded through any suitable groups, for example, sulfonic acid, phosphoric acid, haloalkyl, hydroxyl, halo aldehyde, halosulfonyl, or sulfonamide (—SO₂NH₂) moiety present or modified to be present on the NSAID.

An advantage of the compounds in accordance with embodiments of the invention is that the compounds include a moiety which is a naturally occurring α-amino acid such as L-proline (3), and, unlike most other alkylamines, which is free or substantially free of carcinogenicity. Nitrosation of L-proline would yield N-nitrosoproline (4), which has been the subject of numerous published reports examining the effects of its long-term administration to animals, but so far, none of these studies showed it to be tumorigenic (Hecht et al., Cancer Lett. 1988, 42, 141-145; Lijinsky et al., IARC Sci. Publ 1982, 625-631; Greenblatt et al., J. Natl. Cancer Inst. 1972, 48, 1389-1392; Nagasawa et al., J. Med. Chem. 1973, 16, 583-585; Garcia et al., Z. Krebsforsch. Klin. Onkol. Cancer Res Clin. Oncol. 1973, 79, 141-144; Nixon et al., Food Cosmet. Toxicol. 1976, 14, 133-135; and Mirvish et al., J. Natl. Cancer Inst. 1980, 64, 1435-1442), as well as http://potency.berkeley.edu/.

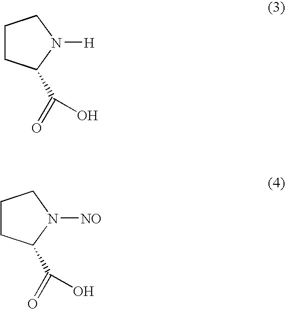

Compounds of Formula I and II are designed to metabolize to the active NSAID moiety and two molecules of nitric oxide under physiological conditions, for example, as depicted below:

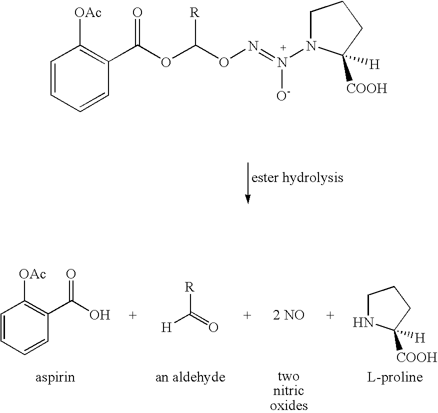

Nitric oxide release from the compounds of Formula I and II can be determined/detected using known techniques such as those described in U.S. Pat. Nos. 6,511,991 and 6,379,660; Keefer, et al., “NONOates(1-Substituted Diazen-1-ium-1, 2 diolates) as Nitric Oxide Donors: Convenient Nitric Oxide Dosage Forms,” Methods in Enzymology, 28: 281-293 (1996); Horstmann et al., “Release of nitric oxide from novel diazeniumdiolates monitored by laser magnetic resonance spectroscopy,” Nitric Oxide, 6(2): 135-41 (2002); and Kitamura et al., “In vivo nitric oxide measurements using a microcoaxial electrode,” Methods Mol. Biol., 279: 35-44 (2004), which are incorporated herein by reference. In general, the amount of NO produced can be detected by a chemiluminescence method, electrochemical method, and/or an absorbance method. In addition, nitric oxide assay kits are commercially available.

In an embodiment, the ability of a compound of Formula I or II to inhibit ovine COX-1 and COX-2 (IC₅₀ value, μM) can be determined using an enzyme immuno assay (EIA) kit (catalog number 560101, Cayman Chemical, Ann Arbor, Mich., USA) according to the method reported by Uddin et al. (Bioorg. Med. Chem. 2004, 12, 5929-5940).

Anti-inflammatory activity can be measured by any method, including the method described by Winter et al. (Proc. Soc. Exp. Biol. Med. 1962, 111, 544-547). For example, compounds of Formula I or II and/or various reference NSAIDs (e.g., aspirin, ibuprofen, and indomethacin) can be evaluated using an in vivo rat carrageenan-induced foot paw edema model (Winter et al., supra).

The ability to produce gastric damage by a compound of Formula I or II can be evaluated according to any suitable procedure (e.g., Cocco et al., Bioorg. Med. Chem. 2004, 12, 4169-4177). For example, ulcerogenic activity (i.e., gastric damage) is evaluated after oral administration of an NSAID or an equivalent amount of a compound of Formula I or II. All drugs are suspended and administered in a dilute (e.g., 1%) methylcellulose solution. Control rats receive oral administration of vehicle. Food, but not water, is removed 24 h before administration of test compounds. Six hours after oral administration of the drug, rats are euthanized in a CO₂ chamber and their stomachs are removed, cut out along the greater curvature of the stomach, gently rinsed with water, and placed on ice. The number and the length of ulcers observed in each stomach were determined using a magnifier lens. The severity of each gastric lesion was measured along its greatest length (1 mm=rating of 1, 1-2 mm=rating of 2, >2 mm=rating according to their length in mm). The “ulcer index” (UI) for each test compound was calculated by adding the total length (L, in mm) of individual ulcers in each stomach, divided by the number of animals in each group (n=4): UI=(L₁+L₂+L₃+L₄)/4

The present invention also provides a pharmaceutical composition comprising at least one compound of Formula I or II and a pharmaceutically acceptable carrier. Any suitable pharmaceutically acceptable carrier can be used within the context of the invention, and such carriers are well known in the art. The choice of carrier will be determined, in part, by the particular site to which the pharmaceutical composition is to be administered and the particular method used to administer the pharmaceutical composition.

Suitable formulations include aqueous and non-aqueous solutions, isotonic sterile solutions, which can contain anti-oxidants, buffers, bacteriostats, and solutes that render the formulation isotonic with the blood or other bodily fluid of the intended recipient, and aqueous and non-aqueous sterile suspensions that can include suspending agents, solubilizers, thickening agents, stabilizers, and preservatives. In one embodiment, the pharmaceutically acceptable carrier is a liquid that contains a buffer and a salt. The formulation can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. In one embodiment, the pharmaceutically acceptable carrier is a buffered saline solution.

Further carriers include sustained-release preparations, such as semipermeable matrices of solid hydrophobic polymers containing the active agent, which matrices are in the form of shaped articles (e.g., films, liposomes, or microparticles).

The pharmaceutical composition can include carriers, thickeners, diluents, buffers, preservatives, surface active agents and the like. The pharmaceutical compositions can also include one or more additional active ingredients, such as antimicrobial agents, anti-inflammatory agents, anesthetics, and the like.

The pharmaceutical composition comprising the compound of Formula I or II can be formulated for any suitable route of administration, depending on whether local or systemic treatment is desired, and on the area to be treated. The pharmaceutical composition can be formulated for parenteral administration, such as intravenous, intraperitoneal, intramuscular, or intratumoral injection. Injectables can be prepared in conventional forms, either as liquid solutions or suspensions, solid forms suitable for suspension in liquid prior to injection, or as emulsions. Additionally, parental administration can involve the preparation of a slow-release or sustained-release system, such that a constant dosage is maintained. Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like.

Desirably, the pharmaceutical composition also can be administered orally. Oral compositions can be in the form of powders or granules, suspensions or solutions in water or non-aqueous media, capsules, sachets, or tablets. Thickeners, flavorings, diluents, emulsifiers, dispersing aids, or binders may be desirable.

Suitable carriers and their formulations are further described in A. R. Gennaro, ed., Remington: The Science and Practice of Pharmacy (19th ed.), Mack Publishing Company, Easton, Pa. (1995).

The pharmaceutical composition can potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

The compound or a pharmaceutical composition comprising at least one compound of Formula I or II can be administered in any suitable manner depending on whether local or systemic treatment is desired, and on the area to be treated. Desirably, the pharmaceutical composition is administered orally, but can be administered parenterally, most preferably by intravenous, intraperitoneal, intramuscular, or intratumoral injection. By the term “injecting,” it is meant that the pharmaceutical composition is forcefully introduced into the target tissue. Although more than one route can be used to administer the pharmaceutical composition, a particular route can provide a more immediate and more effective reaction than another route. For regional delivery, the pharmaceutical composition can be administered intraarterially or intravenously, e.g., via the hepatic artery for delivery to the liver or the carotid artery for delivery to the brain.

The compound or a pharmaceutical composition comprising at least one compound of Formula I or II can be administered in or on a device that allows controlled or sustained release of the compound of Formula I or II, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the active agents. The pharmaceutical compositions of the inventive method also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid. Of course, administration of the compound or pharmaceutical composition can be accomplished via any route that efficiently delivers the active agents to the target tissue.

In another embodiment, the present invention provides a method of preventing or treating cancer comprising administering an effective amount of a compound of the invention. An “effective amount” means an amount sufficient to show a meaningful benefit in an individual, e.g., promoting at least one aspect of tumor cell cytotoxicity, or treatment, healing, prevention, delay of onset, or amelioration of other relevant medical condition(s) associated with a particular cancer.

Effective amounts may vary depending upon the biological effect desired in the individual, condition to be treated, and/or the specific characteristics of the compound of Formula I or II, and the individual. In this respect, any suitable dose of the compound of Formula I or II can be administered to the mammal, according to the type of cancer to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of Formula I or II desirably comprises about 0.1 mg per kilogram (kg) of the body weight of the mammal (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the compound of Formula I or II comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The effectiveness of the treatment of the method of the present invention, in part flows from the effectiveness of NSAIDs in treating various cancers. It has been reported that aspirin, and some other NSAIDs, can significantly reduce colon polyp formation in those at high risk of developing them, including those already treated for colorectal cancer. Chemopreventive effects of aspirin and other NSAIDs can also be seen with cancers of the stomach, esophagus (Thun et al., Cancer Res, 53(6): 1322-7 (1998)), and bladder (Earnest et al., 1992). Aspirin, ibuprofen, piroxicam (Reddy, Cancer Res, (50):2562-2568 (1990); Singh et al., Carcinogenesis, (15): 1317-1323 (1994)), indomethacin (Narisawa et al., Cancer Res, 41(5):1954-7 (1981)), and sulindac (Piazza et al., Cancer Res, (57): 2909-2915 (1997); Rao et al., Cancer Res, (55): 1464 1472 (1995)), effectively inhibit colon carcinogenesis in the AOM-treated rat model and flurbiprofen has demonstrated anti-tumor effects in the APC(Min)+mouse model (Wechter et al., Cancer Res, 57(19): 4316-24 (1997)). NSAIDs also inhibit the development of tumors harboring an activated Ki-ras (Singh and Reddy, Annals of the New York Academy of Sciences, (768): 205-209 (1995)).

Further, it has been found that women who took aspirin seven or more times a week had a 26% lower risk of developing hormone-positive tumors (tumors that are either estrogen receptor (ER) or progesterone receptor (PR)-positive) than women who did not take it. About 60-70% of all breast cancers are hormone positive (Terry et al., JAMA, 291(20): 2433-2440 (2004)).

Additionally, it has been reported that abnormally high levels of COX-2 are found in tumors and premalignant growths of the esophagus, stomach, breast, prostate, lung, bladder, pancreas, skin, cervix, head, and neck as well as the colon and rectum. The link emerged most strongly in colorectal cancer. In the mid-1990s, for example, scientists knocked out the gene for COX-2 in a strain of mice genetically predisposed to cancer of the gut. Without the enzyme, these mice had an 86% reduction in the number of intestinal polyps, the precursors to colorectal cancer. Treatment with a selective COX-2 inhibitor had similar results in the high-risk mice (Reddy et al., Cancer Res, 56: 4566-4569 (1996); Kawamori et al., Cancer Res, 58: 409-412 (1998)). Celecoxib is shown to cause nearly complete suppression of chemically induced colon cancer in rats (Reddy et al., Cancer Res, 60(2): 293-297 (2000))

In addition, COX-2 inhibitors such as nemisulide and nabumetone inhibit formation of colonic aberrant crypt foci in male F344 rats (Rao et al., Proc. Am. Assoc. Cancer Res, 40: 373 (1999)). Additional evidence supporting a tumor-suppressive role for COX-2 comes from studies showing that MF-Tricyclic, a COX-2 inhibitor, blocks intestinal tumorigenesis in APC (DELTA 716) mice (Oshima et al., Cell, 87: 803-809 (1996)).

Accordingly, the present invention provides a method for preventing and/or treating cancer in a mammal. The method comprises administering an effective amount of a compound of Formula I or II to a mammal in need thereof, wherein the compound of Formula I or II prevents or treats the cancer. The term “treating,” as used herein, encompasses all methods of treatment, including treating pre-cancerous symptoms to prevent the onset of cancer, treating primary malignancies to limit, halt, or reverse the tumor growth or to prevent metastasis, and treating tumor cells by causing cell death.

Cancers treatable with the present methods include tumors associated with the oral cavity (e.g., the tongue and tissues of the mouth) and pharynx, the digestive system (e.g., the esophagus, stomach, small intestine, colon, rectum, anus, liver, gall bladder, and pancreas), the respiratory system (e.g., the larynx, lung, and bronchus), bones and joints (e.g., bony metastases), soft tissue, the skin (e.g., melanoma and squamous cell carcinoma), breast, the genital system (e.g., the uterine cervix, uterine corpus, ovary, vulva, vagina, prostate, testis, and penis), the urinary system (e.g., the urinary bladder, kidney, renal pelvis, and ureter), the eye and orbit, the brain and nervous system (e.g., glioma), and the endocrine system (e.g., thyroid). The target tissue also can be located in lymphatic or hematopoietic tissues. For example, the tumor can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). The tumor to be treated is not necessarily the primary tumor. Indeed, the tumor can be a metastasis of a primary tumor located in a different tissue or organ.

Specific examples of cancers treatable with the present methods include, without limitation, acute lymphoblastic leukemia, acute myeloid leukemia, adrenocortical carcinoma, AIDS-related lymphoma, AIDS-related malignancies, anal cancer, cerebellar astrocytoma, extrahepatic bile duct cancer, bladder cancer, osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, chronic lymphocytic, leukemia, chronic myelogenous leukemia, liver cancer, non-small cell lung cancer, small cell lung cancer, Hodgkin's disease, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g. renal pelvis and ureter), retinoblastoma, rhabdomyosarcoma, salivary gland cancer, malignant fibrous histiocytoma of bone, soft tissue sarcoma, sezary syndrome, skin cancer, small intestine cancer, stomach (gastric) cancer, supratentorial primitive neuroectodermal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.

The cancers that will be treatable or preventable by the methods of the present invention include, without limitation, brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

In an embodiment of the method of the invention, the cancer is colon cancer, breast cancer, pancreatic cancer, brain cancer, lung cancer, stomach cancer, a blood cancer, skin cancer, testicular cancer, prostate cancer, ovarian cancer, liver cancer, esophageal cancer, or familial adenomatous polyposis.

In another embodiment, the present invention provides a method for treating individuals exhibiting pre-cancerous symptoms to prevent the onset of cancer, comprising administering a compound of Formula I or II. Cells of this category include those of polyps and other precancerous lesions, premalignancies, preneoplastic or other aberrant phenotype indicating probable progression to a cancerous state.

In yet another embodiment, the present invention provides a method for treating individuals exhibiting primary malignancies to limit, halt, or reverse the tumor growth or to prevent metastasis comprising administering a compound of Formula I or II. Target cancer cells include cancers of the lung, brain, prostate, kidney, liver, ovary, breast, skin, stomach, esophagus, head and neck, testicles, colon, rectum, cervix, lymphatic system, and blood.

In a further embodiment, the individual subject to be treated is a mammal, preferably a human which due to underlying disease or a genetic defect is in risk of developing cancer (e.g., colorectal cancer). For example, humans at risk for developing colorectal cancer include: hereditary nonpolyposis colorectal cancer (HNPCC) patients, polyp patients, patients with a history of colorectal cancer (CRC), individuals over 50 years, who are first-degree relatives of patients with CRC, first-degree relatives of individuals with CRC diagnosed before the age of 50 years, individuals with two first-degree relatives with CRC, and individuals with chronic inflammatory intestinal diseases (e.g., ulcerative colitis and Crohn's disease). Accordingly, in the use and method according to the present invention, the preferred embodiment is one wherein the human is selected from the group being in risk of development of colorectal cancer due to being a first-degree relative to a patient with colorectal cancer, and/or because the individual carries the gene(s) for HNPCC, and/or has familial adenomatous polyposis, colorectal adenomas and/or an inflammatory bowel disease such as ulcerative colitis or Crohn's disease.

In one aspect of the present invention, the method can reduce the risk of developing cancer (e.g., colorectal cancer, breast cancer) in the individual human receiving the treatment by at least 10% or more. The reduction may be at least 20% or more, and in certain circumstances e.g. for high risk patients even 30% or more, preferably about 50%. The period used for measurement can be at least 3 months, such as at least 6 months, most preferred at least 1 year, such as at least 2 years. The effect can be measured, for example, by the number of aberrant crypt foci in AOM induced rats receiving administration of at least one compound of Formula I or II.

In yet another embodiment, the present invention involves the specific killing of tumor cells. Killing can be achieved by apoptotic or non-apoptotic mechanism. The tumor can be a solid tumor or a tumor associated with soft tissue (i.e., soft tissue sarcoma), in a mammal. The term “tumor” refers to both tumor cells and associated stromal cells. The tumor can be associated with the cancers (i.e., located in) described herein.

The tumor can be at any stage. The term “tumor stage” is used in the art to describe the tumor type and the degree of tumor spread. Several tumor staging systems are known in the art, and any suitable staging system can be used to determine the stage of the tumor to be treated. For example, the TNM (Tumor, Node, Metastasis) system is an internationally accepted system that is used frequently to determine tumor stage. In this regard “T” describes the size, depth, and area of the primary tumor. “TX” indicates that the primary tumor cannot be assessed. “T0” indicates that there is no evidence of a primary tumor. “Tis” indicates carcinoma in situ (i.e., the malignant cells are confined to the epithelial layer of the tissue). “T1” indicates a localized tumor two centimeters (cm) or less in diameter and confined to the organ of origin. “T2” indicates a localized tumor less than 5 cm in diameter that extends into adjacent tissue of the same organ. “T3” indicates an advanced tumor greater than 5 cm in diameter with greater involvement of adjacent tissue of the same organ, and “T4” indicates a massive tumor that extends into nerves, blood vessels, bone, or another organ. “N” describes whether the cancer has spread to regional lymph nodes. In this respect, “NX” indicates that regional lymph nodes cannot be assessed. “N0” indicates that there is no evidence of metastases to regional lymph nodes, and stages “N1,” “N2,” and “N3” indicate increasing involvement of regional lymph nodes. The “M” classification describes the presence or absence of distant metastases. In this regard, “MX” indicates that distant metastases cannot be assessed. “M0” indicates that there is no evidence of metastases, and “M1” indicates the presence of distant metastases. Once a tumor has been classified according to the TNM system, each classification can be combined, and an overall stage of I, II, III, or IV can then be assigned to the tumor. While the above description of the TNM system applies generally to most tumor types, the specific definition of each level can vary depending on the type of cancer. Further information about tumor staging is described in, for example, AJCC Cancer Staging Manual, 6th ed., American College of Surgeons, Lippincott-Raven. Philadelphia (2002).

The inventive method also is useful in treating tumors of any grade. The “grade” of a tumor refers to the degree of differentiation of tumor cells. Tumor grade is typically assessed by histological characterization of a tumor sample and determination of the growth rate of the tumor cells (such as by measuring the mitotic index). In general, tumor cells that are well-differentiated resemble normal cells and are of a lower grade (e.g., Grade 1 or 2), while undifferentiated tumor cells are typically more aggressive and are of a higher grade (e.g., Grade 3 or 4).

The tumor can be of any size. Ideally, in treating the mammal for cancer, the inventive method results in cancerous (tumor) cell death and/or reduction in tumor size. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

The tumor can be amenable to surgical removal (i.e., “resection”). In this respect, the inventive method can be used following surgical resection to eliminate any residual tumor cells, prevent growth, or delay the onset of new tumor cells. Alternatively, the target tissue can be a tumor that is surgically unresectable. In this case, the inventive method can be used to affect shrinkage of the tumor, thereby facilitating surgical resection.

In accordance with still another embodiment of the present invention, there is provided a method for treating inflammation and/or an inflammation-related condition in a mammal. Such method comprises administering an effective amount of a compound of the invention to the mammal.

Inflammation-related conditions contemplated for treatment in accordance with the present invention include arthritis (e.g., rheumatoid arthritis, gouty arthritis, osteoarthritis, juvenile arthritis, systemic lupus erythematosus, spondyloarthopathies, and the like), gastrointestinal conditions (e.g., inflammatory bowel disease, Crohn's disease, gastritis, irritable bowel syndrome, ulcerative colitis, and the like), headache (e.g., migraine), asthma, bronchitis, menstrual cramps, tendonitis, and bursitis.

As readily recognized by those of skill in the art, inflammation-related conditions are associated with a variety of conditions, such as, for example, vascular diseases, periarteritis nodosa, thyroidiris, aplastic anemia, Hodgkin's disease, sclerodoma, rheumatic fever, type I diabetes, myasthenia gravis, colorectal cancer, sarcoidosis, nephrotic syndrome, Behcet's syndrome, potymyositis, gingivitis, hypersensitivity, conjunctivitis, swelling occurring after injury, myocardial ischemia, and the like.

In an embodiment of the invention, upon metabolism, nitric oxide and the active NSAID (e.g., aspirin, celecoxib) are simultaneously released. The dual release can prevent thrombus and adverse cardiovascular events, such as myocardial infarction.

The following examples further illustrate the invention but, of course, should not be construed as in any way limiting its scope.

### Example 1

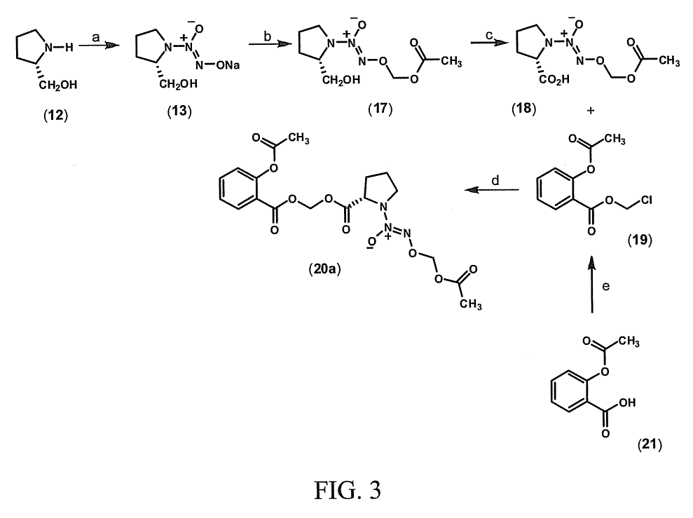

This example demonstrates a method of preparing a compound of Formula II, O²-(acetoxymethyl) 1-[2-(acetylsalicyloyloxymethyloxycarbonyl)pyrrolidin-1-yl]diazen-1-ium-1,2-diolate (20a) (FIG. 3).

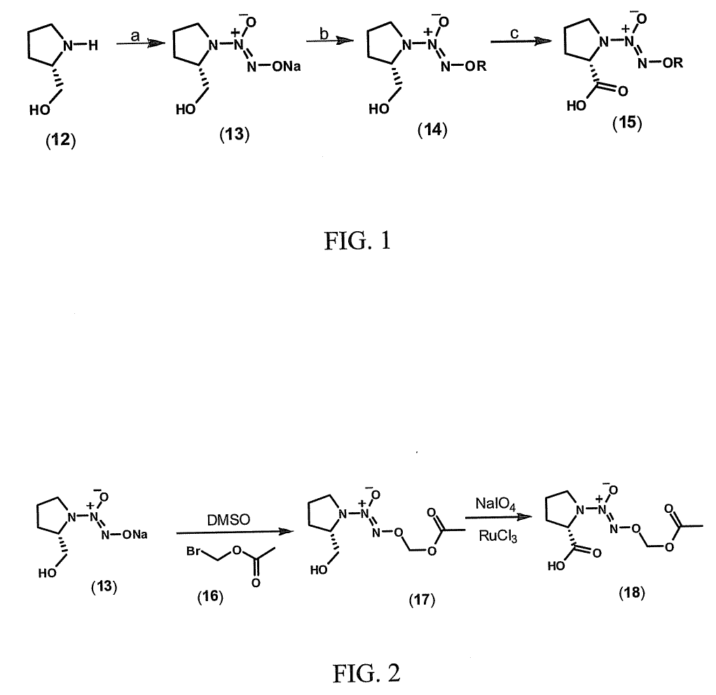

Commercially available L-prolinol (12) is reacted with .NO in the presence of sodium methoxide to yield sodium diazeniumdiolate 13, leaving the adjacent chiral center untouched (FIG. 1). By reacting diazeniumdiolate 13 with bromomethyl acetate (16) in dimethylsulfoxide (DMSO), O²-(acetoxymethyl) 1-[2-(hydroxymethyl)pyrrolidin-1-yl)diazen-1-ium-1,2-diolate (17) is obtained. Compound 17 is oxidized with sodium periodate and ruthenium chloride as catalyst to the corresponding carboxylic acid derivative (18) (FIG. 2).

A mixture of O²-(acetoxymethyl) 1-[2-(hydroxycarbonyl)pyrrolidin-1-yl]diazen-1-ium-1,2-diolate (18, 0.8 g, 3.2 mmol), dimethylsulfoxide (3 mL) and triethylamine (0.3 g, 3.2 mmol) is stirred at 25° C. for five minutes, before adding chloromethyl acetylsalicylate (19, 0.73 g, 3.2 mmol) previously dissolved in dimethylsulfoxide (3 mL) (FIG. 3). After stirring for 24 h at 25° C., the reaction is quenched by adding ethyl acetate (100 mL). The organic phase is washed with water (4×30 mL), dried (MgSO₄), and the solvent is evaporated under vacuum. The residue (1.27 g of a pale yellow liquid) is purified by column chromatography using 80 g Silica Gel 60, and eluted with 2:1 hexanes:ethyl acetate to give 0.91 g of pure product (20a). ¹H-NMR (CDCl₃): δ 2.08 (s, 3H, COCH₃), 2.11 (m, 4H, pyrrolidin-1-yl H-3 and H-5), 2.34 (s, 3H, PhO₂CCH₃), 3.77 (m, 2H, pyrrolidin-1-yl H-5), 4.59 (dd, J=12.9, 5.7 Hz, 1H, pyrrolidin-1-yl H-2), 5.66 (d, J=10.8 Hz, 1H, NO—CH′H—O2C), 5.72 (d, J=10.8 Hz, 1H, NO—CH′H—O₂C), 5.93 (d, J=8.4 Hz, 1H, CO₂CHH′O₂C), 6.04 (d, J=8.4 Hz, 1H, CO₂CHH′O₂C), 7.11 (dd, J=12, 1.5 Hz, 1H, Ph H-3), 7.32 (td, J=11.7, 1.5 Hz, 1H, Ph H-5), 7.60 (td, J=11.7, 2.7 Hz, 1H, Ph H-4), 8.06 (dd, J=11.7, 2.4 Hz, 1H, Ph H-6).

### Example 2

This example demonstrates a method for preparing another compound of Formula II, the synthesis of O²-(acetoxymethyl) 1-{2-[2-[1-(4-chlorobenzoyl)-5-methoxy-2-methyl-1H-indol-3-yl]acetoxymethyloxycarbonyl]pyrrolidin-1-yl}diazen-1-ium-1,2-diolate (20b), in which the NSAID is indomethacin.

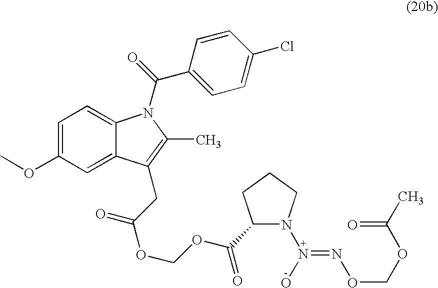

A mixture of O²-(acetoxymethyl) 1-[2-(hydroxycarbonyl)pyrrolidin-1-yl]diazen-1-ium-1,2-diolate (18, 0.8 g, 3.2 mmol), dimethylsulfoxide (3 mL) and triethylamine (3.2 mmol) is stirred at 25° C. for five minutes, before adding chloromethyl 2-[1-(chlorobenzoyl)-5-methoxy-2-methyl-1H-indol-3-yl]acetate (3.2 mmol) previously dissolved in dimethylsulfoxide (3 mL). After stirring for 48 h at 25° C., the reaction is quenched by adding ethyl acetate (100 mL). The organic phase is washed with water (4×30 mL), dried (MgSO₄), and the solvent is evaporated under vacuum. The residue (1.27 g of a pale yellow liquid) is purified by column chromatography using 80 g Silica Gel 60, and eluted with 2:1 hexanes:ethyl acetate to give 0.91 g of pure product (20b). ¹H-NMR (CDCl₃): δ 1.67-2.22 (m, 4H, pyrrolidin-1-yl H3 and H4), 2.10 (s, 3H, COCH₃), 2.36 (s, 3H, CH₃), 3.58-3.81 (m, 2H, pyrrolidin-1-yl H-5), 3.72 (s, 2H, CH₂CO₂), 3.84 (s, 3H, OCH₃), 4.51 (dd, J=13.2, 6.3 Hz, 1H, pyrrolidin-1-yl H-2), 5.68 (d, J=10.8 Hz, 1H, NO—CH′H—O₂C), 5.72 (d, J=10.8 Hz, 1H, NO—CH′H—O2C), 5.75 (d, J=8.4 Hz, 1H, CO₂CHH′O₂C), 5.85 (d, J=8.4 Hz, 1H, CO₂CHH′O₂C), 6.67 (dd, J=13.8, 3.6 Hz, 1H, indol-3-yl H-6), 6.91 (d, J=13.2 Hz, 1H, indol-3-yl H-7), 6.95 (d, J=3.3 Hz, 1H, indol-3-yl H-4), 7.47 (d, J=12.9 Hz, 2H, Ph H-3 and H-5), 7.66 (d, J=12.9 Hz, 2H, Ph H-2 and H-6).

### Example 3

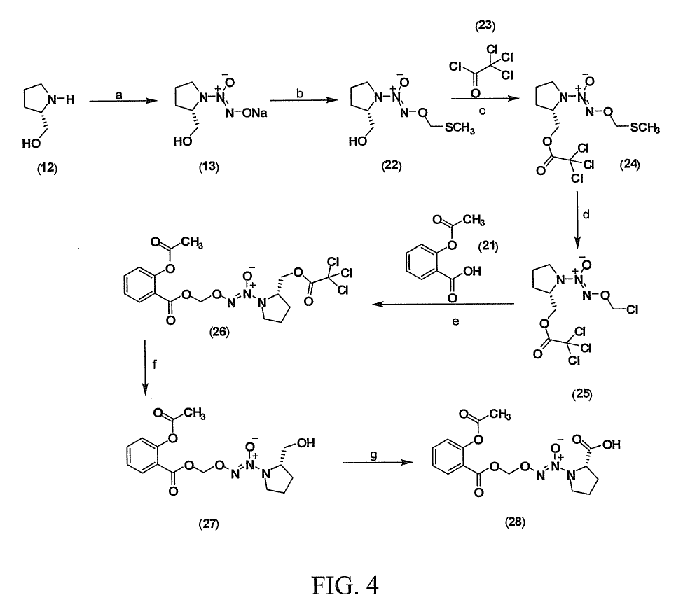

This example demonstrates a method for preparing a compound of Formula I, the synthesis of 28, in which the NSAID is aspirin (FIG. 4).

Commercially available L-prolinol (12) is reacted with .NO in the presence of sodium methoxide to yield sodium diazeniumdiolate 13, leaving the adjacent chiral center untouched (FIG. 1). By reacting diazeniumdiolate 13 with chloromethyl methylsulfide in dimethylformamide (DMF), O²-(methylthiomethyl) 1-[2-(hydroxymethyl)pyrrolidin-1-yl]diazen-1-ium-1,2-diolate (22) is obtained. Compound 22 is reacted with 2,2,2-trichloroacetyl chloride (23) in tetrahydrofuran (THF) and triethylamine (TEA) to provide compound 24. Compound 24 is chlorinated with sulflryl chloride (SO₂Cl₂) in dichloromethane (DCM) to provide 25, which is further reacted with acetylsalicylic acid (21) in DMSO and TEA to provide compound 26. Compound 26 is deprotected with a catalytic amount of K₂CO₃ in CH₃CN/H₂O to provide 27. Compound 27 is oxidized with sodium periodate and ruthenium chloride as catalyst to the corresponding carboxylic acid derivative (28) (FIG. 4).

### Example 4

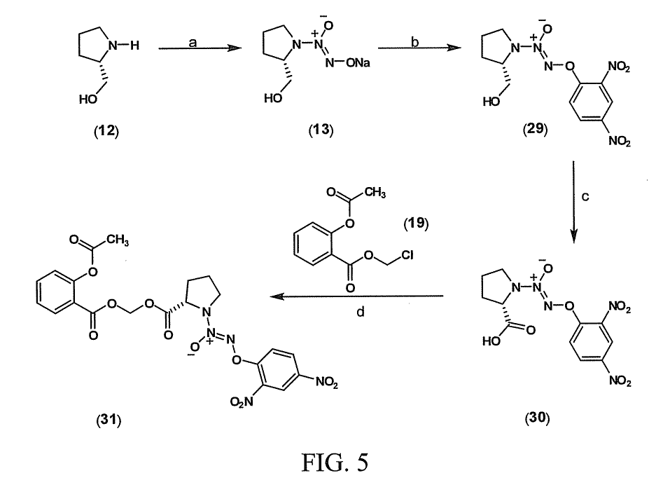

This example demonstrates a method for preparing a compound of Formula II, the synthesis of 31, in which the NSAID is aspirin (FIG. 5).

L-Prolinol (12) is reacted with .NO in the presence of sodium methoxide to yield sodium diazeniumdiolate 13. By reacting diazeniumdiolate 13 with 2,4-dinitro-chlorobenzene (DNCB) in DMF, O²-(2,4-dinitrophenyl) 1-[2-(hydroxymethyl)pyrrolidin-1-yl)diazen-1-ium-1,2-diolate (29) is obtained. Compound 29 is oxidized with sodium periodate and ruthenium chloride as catalyst to the corresponding carboxylic acid derivative 30, which is further reacted with chloromethyl acetylsalicylate (19) (see Example 1) in DMSO and TEA to provide compound 31 (FIG. 5).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of Formula I 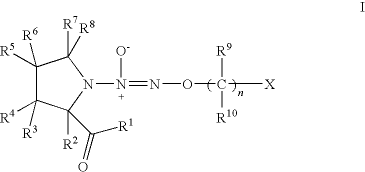
wherein
R¹ is hydrogen, OH, halo, NR¹¹R¹², OR¹¹, SR¹¹, or OM_{1/m}, wherein M is a cation and m is the valency of M,
R², R⁷, and R⁸ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, carboxy, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, or nitro;
R³⁻⁶ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, hydroxy, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, mercapto, an unsubstituted or substituted C₁₋₁₂ alkylthio, amino, an unsubstituted or substituted C₁₋₁₂ alkylamino, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, halo, or nitro;
R⁹ and R¹⁰ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, an unsubstituted or substituted heteroarylamino, an unsubstituted or substituted heteroaryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, or benzylcarbonyl;
X is a functional portion of an NSAID;
R¹¹ and R¹² are independently hydrogen, an unsubstituted or substituted C₁₋₁₂ alkyl, an unsubstituted or substituted C₂₋₁₂ alkenyl, an unsubstituted or substituted C₃₋₃₀ cycloalkyl, an unsubstituted or substituted C₆₋₃₀ aryl, an unsubstituted or substituted heteroaryl, or an unsubstituted or substituted heterocyclyl; and
n is 0-3, or a pharmaceutically acceptable salt thereof.

2. A compound of Formula II 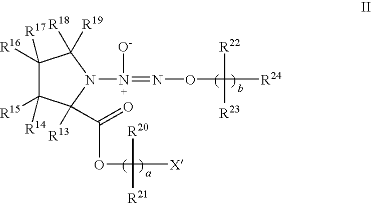
wherein
X′ is a functional portion of an NSAID;
R¹³, R¹⁸, and R¹⁹ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, carboxy, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, or nitro;
R¹⁴⁻¹⁷ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, hydroxy, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, mercapto, an unsubstituted or substituted C₁₋₁₂ alkylthio, amino, an unsubstituted or substituted C₁₋₁₂ alkylamino, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, benzylcarbonyl, cyano, halo, or nitro;
R²⁰⁻²³ are the same or different and each is independently hydrogen, an unsubstituted or substituted C₁₋₁₂ alkyl, an unsubstituted or substituted C₂₋₁₂ alkenyl, an unsubstituted or substituted C₂₋₁₂ alkynyl, an unsubstituted or substituted C₃₋₃₀ cycloalkyl, an unsubstituted or substituted C₆₋₃₀ aryl, an unsubstituted or substituted aralkyl, an unsubstituted or substituted heteroaryl, an unsubstituted or substituted heterocyclyl, an unsubstituted or substituted C₁₋₁₂ alkoxy, an unsubstituted or substituted C₆₋₃₀ aryloxy, an unsubstituted or substituted heteroaryloxy, an unsubstituted or substituted aralkyloxy, an unsubstituted or substituted C₁₋₁₂ alkylthio, an unsubstituted or substituted C₆₋₃₀ arylamino, an unsubstituted or substituted C₁₋₁₂ dialkylamino, an unsubstituted or substituted C₆₋₃₀ diarylamino, an unsubstituted or substituted C₆₋₃₀ aryl-C₁₋₁₂ alkylamino, an unsubstituted or substituted heteroarylamino, an unsubstituted or substituted heteroaryl-C₁₋₁₂ alkylamino, carboxy, an unsubstituted or substituted carboxy-C₁₋₁₂ alkylamino, an unsubstituted or substituted carboxy-C₁₋₁₂ dialkylamino, carboxamido, an unsubstituted or substituted C₁₋₁₂ alkylcarboxamido, an unsubstituted or substituted C₁₋₁₂ dialkylcarboxamido, an unsubstituted or substituted carboxy-C₁₋₁₂ alkyl, an unsubstituted or substituted C₁₋₁₂ alkylcarbonyl, C₇₋₃₁ aroyl, or benzylcarbonyl;
R²⁴ is an unsubstituted or substituted C₁₋₁₂ acyloxy, an unsubstituted or substituted carboxamido, an unsubstituted or substituted C₁₋₁₂ alkyl, an unsubstituted or substituted C₂₋₁₂ alkenyl, an unsubstituted or substituted C₃₋₃₀ cycloalkyl, an unsubstituted or substituted C₆₋₃₀ aryl, or an unsubstituted or substituted C₁₋₁₂ alkoxy-C₁₋₁₂ alkyl; and
a and b are independently 0-3, or a pharmaceutically acceptable salt thereof.

3. The compound or salt of claim 1, wherein R¹ is hydrogen, OH, OR¹¹, or OM_{1/m}.

4. The compound or salt of claim 1, wherein
R², R⁷, and R⁸ are individually selected from hydrogen, an unsubstituted or substituted C₁₋₁₂ alkyl, an unsubstituted or substituted C₆₋₃₀ aryl, and an unsubstituted or substituted C₁₋₁₂ alkoxy; and
R³⁻⁶ are individually selected from hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo.

5. The compound or salt of claim 1, wherein R⁹ and R¹⁰ are individually selected from hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl.

6. The compound or salt of claim 1, wherein the NSAID is aspirin, ibuprofen, sulindac, indomethacin, or celecoxib.

7. The compound or salt of claim 1, wherein
R¹ is hydrogen, OH, OR¹¹, or OM_{1/m};
R², R⁷, and R⁸ are individually selected from hydrogen, an unsubstituted or substituted C₁₋₁₂ alkyl, an unsubstituted or substituted C₆₋₃₀ aryl, and an unsubstituted or substituted C₁₋₁₂ alkoxy;
R³⁻⁶ are individually selected from hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo;
R⁹ and R¹⁰ are individually selected from hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl; and
the NSAID is aspirin, ibuprofen, sulindac, indomethacin, or celecoxib.

8. The compound or salt of claim 1, wherein R¹ is OH, R²⁻⁸ are hydrogen, R⁹ and R¹⁰ are hydrogen and phenyl, respectively, and n is 1.

9. The compound or salt of claim 1, wherein the compound is

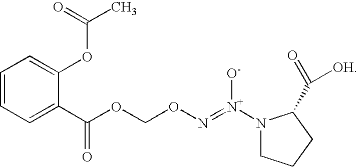

10. The compound or salt of claim 2, wherein
R¹³, R¹⁸, and R¹⁹ are individually selected from the group consisting of hydrogen, an unsubstituted or substituted C₁₋₁₂ alkyl, an unsubstituted or substituted C₆₋₃₀ aryl, and an unsubstituted or substituted C₁₋₁₂ alkoxy; and
R¹⁴⁻¹⁷ are individually selected from the group consisting of hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo.

11. The compound or salt of claim 2, wherein R²⁰⁻²³ are individually selected from the group consisting of hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl.

12. The compound or salt of claim 2, wherein R²⁴ is an unsubstituted or substituted C₁₋₁₂ acyloxy, an unsubstituted or substituted C₁₋₁₂ alkyl, an unsubstituted or substituted C₆₋₃₀ aryl, or an unsubstituted or substituted C₁₋₁₂ alkoxy-C₁₋₁₂ alkyl.

13. The compound or salt of claim 2, wherein the NSAID is aspirin, ibuprofen, sulindac, indomethacin, or celecoxib.

14. The compound or salt of claim 2, wherein
R¹³, R¹⁸, and R¹⁹ are individually selected from the group consisting of hydrogen, an unsubstituted or substituted C₁₋₁₂ alkyl, an unsubstituted or substituted C₆₋₃₀ aryl, and an unsubstituted or substituted C₁₋₁₂ alkoxy;
R¹⁴⁻¹⁷ are individually selected from the group consisting of hydrogen, an unsubstituted or substituted C₁₋₁₂ alkyl, an unsubstituted or substituted C₆₋₃₀ aryl, hydroxy, an unsubstituted or substituted C₁₋₁₂ alkoxy, and halo;
R²⁰⁻²³ are individually selected from the group consisting of hydrogen, an unsubstituted or substituted C₁₋₁₂ alkyl, an unsubstituted or substituted C₃₋₃₀ cycloalkyl, an unsubstituted or substituted C₆₋₃₀ aryl, an unsubstituted or substituted heteroaryl, and an unsubstituted or substituted heterocyclyl;
R²⁴ is an unsubstituted or substituted C₁₋₁₂ acyloxy, an unsubstituted or substituted C₁₋₁₂ alkyl, an unsubstituted or substituted C₆₋₃₀ aryl, or an unsubstituted or substituted C₁-12 alkoxy-C₁₋₁₂ alkyl; and
the NSAID is aspirin, ibuprofen, sulindac, indomethacin, or celecoxib.

15. The compound or salt of claim 2, wherein R¹³⁻¹⁹ are hydrogen, R²⁰⁻²³ are individually hydrogen, methyl, ethyl, i-propyl, t-butyl, or phenyl, R²⁴ is an unsubstituted or substituted C₁₋₁₂ acyloxy, a and b are each 1, and the NSAID is aspirin, ibuprofen, sulindac, indomethacin, or celecoxib.

16. The compound or salt of claim 15, wherein R¹³⁻²³ are hydrogen, R²⁴ is methylcarboxy or phenylcarboxy, and the NSAID is aspirin.

17. The compound or salt of claim 15, which is wherein the compound is

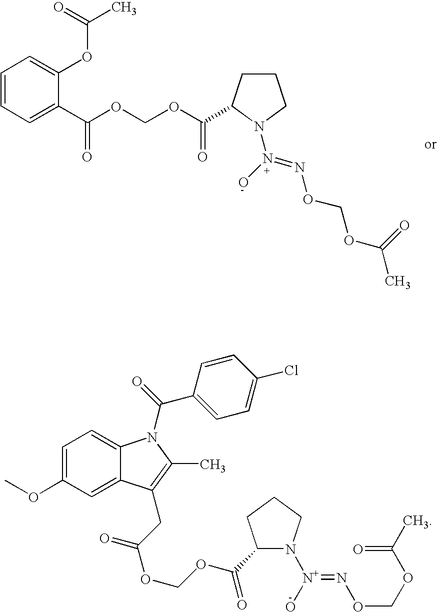

18. The compound or salt of claim 15, wherein R¹³⁻²³ are hydrogen, R²⁴ is methylcarboxy or phenylcarboxy, and the NSAID is indomethacin.

19. (canceled)

20. The compound or salt of claim 2, wherein R¹³⁻²¹ are hydrogen, R²⁴ is an unsubstituted or substituted C₆₋₃₀ aryl, a is 1, b is 0, and the NSAID is indomethacin or aspirin.

21. The compound or salt of claim 20, which is wherein the compound is

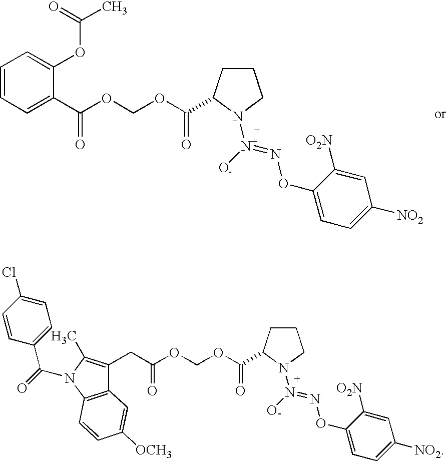

22. (canceled)

23. A pharmaceutical composition comprising at least one compound or salt of claim 1 and a pharmaceutically acceptable carrier.

24. A method of preventing or treating cancer or treating pre-cancerous symptoms or primary malignancies in a mammal comprising administering an effective amount of at least one compound or salt of claim 1 to the mammal.

25.-26. (canceled)

27. A method for treating inflammation or an inflammation-related condition in a mammal comprising administering an effective amount of at least one compound or salt of claim 1 to the mammal.

28. The method according to claim 27, wherein the inflammation-related condition is arthritis.

29. The method according to claim 28, wherein the arthritis is rheumatoid arthritis, gouty arthritis, osteoarthritis, juvenile arthritis, systemic lupus erythematosus, or spondyloarthopathies.

30. The method according to claim 27, wherein the inflammation-related condition is gastrointestinal condition, headache, asthma, bronchitis, menstrual cramps, tendonitis, or bursitis.

31. The method according to claim 30, wherein the gastrointestinal condition is inflammatory bowel disease, Crohn's disease, gastritis, irritable bowel syndrome, or ulcerative colitis.

32. The method according to claim 27, wherein inflammation-related condition is associated with vascular disease, periarteritis nodosa, thyroidiris, aplastic anemia, Hodgkin's disease, sclerodoma, rheumatic fever, type I diabetes, myasthenia gravis, colorectal cancer, sarcoidosis, nephrotic syndrome, Behcet's syndrome, potymyositis, gingivitis, hypersensitivity, conjunctivitis, swelling occurring after injury, and myocardial ischemia.

33. A pharmaceutical composition comprising at least one compound or salt of claim 2 and a pharmaceutically acceptable carrier.

34. A method of preventing or treating cancer or treating pre-cancerous symptoms or primary malignancies in a mammal comprising administering an effective amount of at least one compound or salt of claim 2 to the mammal.

35. A method for treating inflammation or an inflammation-related condition in a mammal comprising administering an effective amount of at least one compound or salt of claim 2 to the mammal.
